(19) United States
(12) Patent Application Publication
(10) Pub. No.: US 2023/0020528 A1
(43) Pub. Date: Jan. 19, 2023

(54) AUTOMATED SYSTEMS AND METHODS FOR PRODUCING DENTAL ALIGNERS

(71) Applicant: David Navarro Guerra, Irving, TX (US)

(72) Inventor: David Navarro Guerra, Irving, TX (US)

(21) Appl. No.: 17/375,721

(22) Filed: Jul. 14, 2021

(51) Int. Cl. A61C 7/08; A61C 7/00; A61C 9/00; B33Y 80/00

(52) U.S. Cl. CPC A61C 7/08; A61C 7/002; A61C 9/0006; A61C 9/0053; B33Y 80/00; A61C 2007/004

(57) ABSTRACT

Systems and methods for automating the production of dental aligners is disclosed. In some embodiments the systems and methods include a three-dimensional printer in communication with one or more computer and one or more computer programs. The system and method configured to permit persons to automatically scan and upload a pre-existing digital scan, x-ray, physical mold or other impression of a person's teeth to the three-dimensional printer for direct real-time printing of custom dental aligners. In some embodiments, the system and method also includes an intraoral scanner configured to permit persons to take and receive a real-time digital scan of their teeth and receive a real-time custom dental aligner, on the same day and from the same integrated system, method, machine, kiosk and location.

### TECHNICAL FIELD

The present disclosure relates generally to the technical field of systems and methods for producing dental aligners and more specifically to, automated systems and methods for same-day dental scanning and same-day three-dimensional printing of dental aligners from a single location, such as from an automated self-serve kiosk and the like.

### BACKGROUND

Traditional dental braces are often prescribed by a licensed dentist or orthodontist and use metal brackets semi-permanently affixed to a person's teeth and connected by metal wires. The wires are tightened or loosed in accordance with a dental plan to gradually force the teeth to move into a desired position to help improve a person's smile, bite, hygiene and for various other reasons. Traditional dental aligners are also often prescribed by a licensed dentist or orthodontist but use a series of tight-fitting custom-made clear mouth pieces that slip over the teeth; but that are not permanently secured thereto. Traditional dental aligners are configured to be selectively removed for eating, brushing, and flossing, for example. Because dental aligners are formed with a pre-set shape custom fit to a person's mouth and teeth, a person often requires a series of different dental aligners be produced, as the person's teeth move and reposition over time, and in accordance with a dental alignment plan proposed by a licensed dentist or orthodontist, to re-position the teeth over time into proper alignment.

Because traditional dental aligners are made to custom-fit a person's teeth, detailed information regarding the position of a person's teeth and size of mouth is needed, often requiring x-rays or a mold be made of a person's mouth and teeth, and various other precise measurements be taken so that each dental aligner will custom fit a particular person's teeth and move the teeth into the desired alignment, Unfortunately, however, this process typically requires a person to physically visit the dentist, orthodontist or aligner production facility multiple times, first to take the appropriate measurements and scans and then to receive or pick up the dental aligner. This process is often made more time consuming because the re-alignment process requires multiple dental aligners to be constructed over a period of time, often six months to a year or more, each time requiring the person to schedule multiple visits, to continuously take new molds, x-rays, scans and other measurements of a person's teeth and mouth and to receive new custom fit dental aligners periodically as the person's teeth are re-aligned. This process is time consuming, unpleasant, cumbersome, inefficient and expensive. As a result, many people that would otherwise benefit from a dental aligner are often hesitant to receive such treatment and undergo the cost and burden associated with such a lengthy and time-consuming process.

To that end it would be advantageous to provide an improved system and method for automating the production of dental aligners. The improved system and method configured to permit persons to receive a digital dental scan of their teeth or scan and upload a pre-existing three-dimensional rendering of their teeth (such as an x-ray, physical mold, or impression) and receive a dental aligner, on the same day, all from the same integrated method, machine, system, kiosk or location and without having to make multiple trips to a dental office or other location to receive the custom fit dental aligner. Thus, providing significant cost and convenience advantages over traditional methods and systems for manufacturing dental aligners. Thereby, improving the health and wellness of persons in need of dental alignment treatment, expanding access to such treatment, and reducing the cost associated therewith.

It is to such improved automated systems and methods that exemplary embodiments of the inventive concepts disclosed and claimed herein are directed.

### SUMMARY

Systems and methods for automated same-day dental scanning and dental aligner printing are disclosed. In one embodiment, a system for automated dental aligner printing is disclosed. The system includes a three-dimensional printer in communication with one or more computers running one or more computer programs. The three-dimensional printer for automated same-day printing of a dental aligner produced from a pre-existing three-dimensional rendering of a user's teeth, such as an x-ray, digital scan, physical mold or other physical impression. The three-dimensional printer having a hot end, an extruder, a print bed and a cooling fan. The computer including a display screen, one or more processors, one or more transceivers for transmitting and receiving wireless data and one or more data storage devices. The computer or three-dimensional printer also including a means for scanning and uploading the three-dimensional rendering of a user's teeth. The means for scanning and uploading may be a combination of scanners, computers, or digital cameras configured for capturing and uploading to the computer the scan, x-ray or a digital representation of the three-dimensional object, including any measurement data associated therewith. For example, the means for scanning and uploading may be positioned within a shelf or opening of a housing of the system such that a user can physically insert a traditional physical mold or a traditional pre-existing dental aligner directly into the shelf or opening of the housing and the physical mold or pre-existing dental aligner can be scanned by the means for scanning and uploading to create a digital representation of the physical mold or pre-existing dental aligner (including any measurement data associated therewith) on one or more computer for production of a new dental aligner. The computer executing a software program for automated same-day scanning of the three-dimensional rendering of a user's teeth and for automated same-day dental aligner printing from the three-dimensional printer. The software program including automated instructions for: receiving a three-dimensional rendering of a user's teeth; generating a three-dimensional digital model of a dental aligner based upon the three-dimensional rendering of a user's teeth; and printing from the three-dimensional printer, automatically and on the same day as the three-dimensional rendering of a user's teeth is received, a dental aligner, based upon the real-time three-dimensional digital model.

In another embodiment, the system includes a three-dimensional printer in communication with one or more computers running one or more computer programs and an intraoral scanner for producing a real-time three-dimensional digital scan of a user's teeth. The intraoral scanner including a scanner, one or more processors, one or more transceivers for transmitting and receiving wireless data and one or more data storage devices. The three-dimensional printer configured for automated same-day printing of a dental aligner produced from the real-time three-dimensional digital scan of a user's teeth. The three-dimensional printer including a hot end, an extruder, a print bed and a cooling fan. The computer including a display screen, one or more processors, one or more transceivers for transmitting and receiving wireless data and one or more data storage devices. The computer executing a software program for automated same-day dental scanning from the intraoral scanner and automated same-day dental aligner printing from the three-dimensional printer. The software program including automated instructions for: receiving a real-time three-dimensional digital scan of a user's teeth from the intraoral scanner; generating a three-dimensional digital model of a dental aligner based upon the real-time three-dimensional digital scan of a user's teeth; and printing from the three-dimensional printer, automatically and on the same day as the three-dimensional digital scan of a user's teeth, a dental aligner based upon the real-time three-dimensional digital model.

In some embodiments, the software program for automated same-day dental scanning from the intraoral scanner and automated same-day dental aligner printing from the three-dimensional printer, further includes automated instructions for: determining a treatment plan for optimal positioning of the dental aligner based upon artificial intelligence. Further, in some embodiments the treatment plan for optimal positioning of the dental aligner is determined by a licensed dentist or is approved by a licensed dentist (on the same day or at a later date) in combination with the software program and one or more computers. In some embodiments, the licensed dentist may be available on-site, while in other embodiments the licensed dentist may be off-site but available via one or more computers or video monitors, for example. Likewise, in some embodiments, development of or approval of the treatment plan may be fully automated by artificial intelligence associated with the system. Further, in some embodiments, the intraoral scanner, three-dimensional printer and computer are each physically housed within a self-serve kiosk. In some embodiments, the computer is in wireless communication with a remote computer or mobile device associated with a user. The computer configured to send automatic notifications and alerts to the remote computer or mobile device concerning the status, same day pick-up time, and cost associated with printing the dental aligner from the system. In some embodiments, the intraoral scanner may be a stand-alone intraoral scanner detachably connected to a remote computer or mobile device and in communication with the computer.

In some embodiments, the computer is in wireless communication with a remote computer associated with a self-serve kiosk. The self-serve kiosk including an Intraoral scanner for producing a real-time three-dimensional digital scan of a user's teeth. The intraoral scanner including a scanner, one or more processors, one or more transceivers for transmitting and receiving wireless data and one or more data storage devices. Further, the self-serve kiosk includes a three-dimensional printer in communication with the intraoral scanner. The three-dimensional printer for automated same-day printing of a dental aligner produced from the real-time three-dimensional digital scan of a user's teeth. The three-dimensional printer including a hot end, an extruder, a print bed and a cooling fan. The self-serve kiosk also including a computer in communication with the intraoral scanner and in communication the three-dimensional printer, the computer including a display screen, one or more processors, one or more transceivers for transmitting and receiving wireless data and one or more data storage devices. The computer executing a software program for automated same-day dental scanning from the intraoral scanner and automated same-day dental aligner printing from the three-dimensional printer. The software program including automated instructions for: receiving a real-time three-dimensional digital scan of a user's teeth from the intraoral scanner; generating a three-dimensional digital model of a dental aligner based upon the real-time three-dimensional digital scan of a user's teeth; and printing from the three-dimensional printer, automatically and on the same day as the three-dimensional digital scan of a user's teeth, a dental aligner based upon the real-time three-dimensional digital model.

Methods for automated same-day dental scanning and dental aligner printing are also disclosed. In some embodiments, a method of dental aligner printing includes the steps of: receiving a three-dimensional rendering of a user's teeth on a computer having a display screen, one or more processors, one or more transceivers for transmitting and receiving wireless data, one or more data storage devices and a means for scanning and uploading a three-dimensional rendering of a user's teeth, the computer executing a software program for automated dental aligner printing from a three-dimensional printer, the three-dimensional printer having a hot end, an extruder, a print bed and a cooling fan; generating a three-dimensional digital model of a dental aligner based upon the three-dimensional rendering of a user's teeth, on a computer having a display screen, one or more processors, one or more transceivers for transmitting and receiving wireless data, one or more data storage devices and a means for scanning and uploading a three-dimensional rendering of a user's teeth, the computer executing a software program for automated same-day dental scanning from the intraoral scanner and automated same-day dental aligner printing from the three-dimensional printer; and printing a dental aligner based upon the real-time three-dimensional digital model from the three-dimensional printer, the three-dimensional printer having a hot end, an extruder, a print bed and a cooling fan, automatically and on the same day the three-dimensional rendering of a user's teeth is received by the computer.

In some embodiments, a method of automated same-day dental scanning and dental aligner printing includes the step of: receiving on a computer having a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a real-time three-dimensional digital scan of a user's teeth from an intraoral scanner. The intraoral scanner including, a scanner, one or more processors, one or more transceivers for transmitting and receiving wireless data and one or more data storage devices. The method also includes the step of: generating on a computer comprising a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a three-dimensional digital model of a dental aligner based upon the real-time three-dimensional digital scan of a user's teeth. The method also includes the step of: printing from a three-dimensional printer having a hot end, an extruder, a print bed and a cooling fan, automatically and on the same day as the three-dimensional digital scan of a user's teeth, a dental aligner based upon the real-time three-dimensional digital model.

In some embodiments, the method also includes the step of: generating on a computer having a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a treatment plan for optimal positioning of the dental aligner based upon artificial intelligence. Further, in some embodiments the treatment plan for optimal positioning of the dental aligner is determined by a licensed dentist or is approved by a licensed dentist (on the same day or at a later date) in combination with the software program and one or more computers. In some embodiments, the licensed dentist may be available on-site, while in other embodiments the licensed dentist may be off-site but available via one or more computers or video monitors, for example. Likewise, in some embodiments, development of or approval of the treatment plan may be fully automated by artificial intelligence associated with the system. Further, in some embodiments, the method includes the step of: sending from a computer having a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a real-time notification that the dental aligner has been printed from the three-dimensional printer and is ready for same-day pick-up, to a mobile device associated with a user.

In some embodiments, the method also includes the step of: displaying on a display screen of a computer having a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a three-dimensional digital model of the dental aligner based upon the real-time three-dimensional digital scan of a user's teeth. Further, in some embodiments, the method includes the step of: calculating on a computer having a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, based upon artificial intelligence, optimal positioning of the dental aligner in a mouth of a user.

In some embodiments, a method of automated dental scanning and dental aligner printing is disclosed. The method includes the steps of: providing an intraoral scanner to a user, the intraoral scanner comprising one or more processors, one or more transceivers for transmitting and receiving wireless data and one or more data storage devices; receiving on the intraoral scanner, a real-time three-dimensional digital scan of the user's teeth; receiving the real-time three-dimensional digital scan of the user's teeth; printing a dental aligner from a three-dimensional printer, the dental aligner based upon the three-dimensional digital scan of the user's teeth; and providing the printed dental aligner to the user. In some embodiments of the method, the intraoral scanner is mailed to the user with instructions for use, such that the user can perform the intraoral scan at home; while in other embodiments, the user may schedule a time to have a professional, such as a licensed dentist or other dental professional visit the user's home to perform the intraoral scan. Once the digital scan is complete, the intraoral scanner may be mailed to a designated location for printing of the dental aligner and then the printed dental aligner may be mailed to the user's home within twenty-four to forty-eight hours, for example. In some embodiments, after a digital scan is made of the user's teeth using the intraoral scanner, the user may elect to travel to a self-serve kiosk (as described herein) where the intraoral scanner and/or the three-dimensional digital scan of the user's teeth may be returned and uploaded to the kiosk for direct printing of the dental aligner from the three-dimensional printer associated with the kiosk. The dental aligner may be printed at the kiosk and picked up by the user within twenty-four to forty-eight hours for example, or the user may elect to have the dental aligner mailed directly to the user's home, or the user may schedule printing of the dental aligner for a certain date/time such that the dental aligner may be picked up or delivered to the user at a date and time selected by the user.

Various other embodiments and aspects of the disclosure will become apparent based on the drawings and detailed description set forth herein.

### BRIEF DESCRIPTION OF THE DRAWINGS

Like reference numerals in the figures represent and refer to the same or similar element or function. Implementations of the disclosure may be better understood when consideration is given to the following detailed description thereof. Such description makes reference to the annexed pictorial illustrations, schematics, graphs, drawings, and appendices. In the drawings:

FIG. 1 is a depiction of an embodiment of a system (100) for automated same-day dental scanning and dental aligner printing according to the inventive concepts disclosed herein.

FIG. 2 is a depiction of a three-dimensional digital scan (125) of a user's teeth in accordance with an embodiment of a system (100) for automated same-day dental scanning and dental aligner printing according to the inventive concepts disclosed herein.

FIG. 3 is a depiction of a dental aligner (111) produced in accordance with an embodiment of a system (100) for automated same-day dental scanning and dental aligner printing according to the inventive concepts disclosed herein.

FIG. 4 is a depiction of a self-serve kiosk (299) for automated same-day dental scanning and dental aligner printing according to the inventive concepts disclosed herein.

FIG. 5 is a depiction of an embodiment of a method (600) for automated same-day dental scanning and dental aligner printing, according to the inventive concepts disclosed herein.

FIG. 6 is a depiction of an embodiment of a system (100) for automated same-day dental scanning and dental aligner printing according to the inventive concepts disclosed herein.

FIG. 7 is a depiction of an embodiment of a self-serve kiosk (299) for automated same-day dental scanning and dental aligner printing according to the inventive concepts disclosed herein.

### DETAILED DESCRIPTION

Before explaining at least one embodiment of the inventive concepts disclosed herein in detail, it is to be understood that the inventive concepts are not limited in their application to the details of construction and the arrangements of the components or steps or methodologies set forth in the following description or illustrated in the drawings. The inventive concepts disclosed herein are capable of other embodiments or of being practiced or carried out in various ways. Also, it is to be understood that the phraseology and terminology employed herein is for the purpose of description and should not be regarded as limiting the inventive concepts claimed herein in any way.

In the following detailed description of embodiments of the inventive concepts, numerous specific details are set forth in order to provide a more thorough understanding of the inventive concepts. However, it will be apparent to one of ordinary skill in the art that the inventive concepts within the disclosure may be practiced without these specific details. In other instances, well-known features have not been described in detail to avoid unnecessarily complicating the instant disclosure.

As used herein, the terms “comprises,” “comprising,” “includes,” “including,” “has,” “having,” or any other variation thereof, are intended to cover a non-exclusive inclusion. For example, a system, process, method, article, or apparatus that comprises a list of elements is not necessarily limited to only those elements but may include other elements not expressly listed.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

The inventive concepts disclosed herein are directed to systems and methods for automated same-day dental scanning and dental aligner printing from a machine, such as a self-serve kiosk or the like. A user may, without the necessity of making an appointment, stop by the kiosk and upload a pre-existing three-dimensional rendering of a person's teeth or receive, on the spot and in real-time a digital scan of the user's teeth and a corresponding custom fit dental aligner constructed from the three-dimensional rendering or digital scan and printed from a three-dimensional printer associated with the kiosk.

In an embodiment, a system for automated dental aligner printing is disclosed. In some embodiments the system is configured to produce a dental aligner on the same day as the dental scan (i.e., production of the dental aligner is configured to be completed and delivered in a single day from a single location); while in other embodiments the system is configured to produce a dental aligner at a different day as the dental scan (i.e., production of the dental aligner may be completed or delivered over the course of several days) for example. The system includes a three-dimensional printer in communication with one or more computers running one or more computer programs. The three-dimensional printer for automated same-day printing of a dental aligner produced from a three-dimensional rendering of a user's teeth. The three-dimensional printer having a hot end, an extruder, a print bed and a cooling fan. The computer including a display screen, one or more processors, one or more transceivers for transmitting and receiving wireless data and one or more data storage devices. The computer and in some embodiments the three-dimensional printer also including a means for scanning and uploading the three-dimensional rendering of a user's teeth. For example, the means for scanning and uploading may be positioned within a shelf or opening of a housing of the system such that a user can physically insert a traditional physical mold or a traditional pre-existing dental aligner directly into the shelf or opening of the housing and the physical mold or pre-existing dental aligner can be scanned by the means for scanning and uploading to create a digital representation of the physical mold or pre-existing dental aligner (including any measurement data associated therewith) on one or more computer for production of a new dental aligner. The means for scanning and uploading may be a combination of scanners, computers, or digital cameras configured for capturing and uploading to the computer the scan, x-ray or a digital representation of the three-dimensional object, including any measurement data associated therewith. The computer executing a software program for automated same-day scanning of the three-dimensional rendering of a user's teeth and for automated same-day dental aligner printing from the three-dimensional printer. The software program including automated instructions for: receiving a three-dimensional rendering of a user's teeth: generating a three-dimensional digital mod& of a dental aligner based upon the three-dimensional rendering of a user's teeth; and printing from the three-dimensional printer, automatically and on the same day as the three-dimensional rendering of a user's teeth is received, a dental aligner, based upon the real-time three-dimensional digital model.

In some embodiments the system also includes an intraoral scanner for producing a real-time three-dimensional digital scan of a user's teeth. The intraoral scanner including a scanner, one or more processors, one or more transceivers for transmitting and receiving wireless data and one or more data storage devices. The system also includes a three-dimensional printer in communication with the intraoral scanner. The three-dimensional printer for automated same-day printing of a dental aligner produced from the real-time three-dimensional digital scan of a user's teeth. The three-dimensional printer having a hot end, an extruder, a print bed and a cooling fan. The system further including a computer in communication with the intraoral scanner and in communication the three-dimensional printer. The computer having a display screen, one or more processors, one or more transceivers for transmitting and receiving wireless data and one or more data storage devices. The computer executing a software program for automated same-day dental scanning from the intraoral scanner and automated same-day dental aligner printing from the three-dimensional printer. The software program having automated instructions for: receiving a real-time three-dimensional digital scan of a user's teeth from the intraoral scanner; generating a three-dimensional digital model of a dental aligner based upon the real-time three-dimensional digital scan of a user's teeth; and printing from the three-dimensional printer, automatically and on the same day as the three-dimensional digital scan of a user's teeth, a dental aligner based upon the real-time three-dimensional digital model.

Methods for automated same-day dental scanning and dental aligner printing are also disclosed. In some embodiments, the method includes the step of: receiving on a computer having a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a real-time three-dimensional digital scan of a user's teeth from an intraoral scanner, the intraoral scanner having, a scanner, one or more processors, one or more transceivers for transmitting and receiving wireless data and one or more data storage devices; generating on a computer having a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a three-dimensional digital model of a dental aligner based upon the real-time three-dimensional digital scan of a user's teeth; and printing from a three-dimensional printer having a hot end, an extruder, a print bed and a cooling fan, automatically and on the same day as the three-dimensional digital scan of a user's teeth, a dental aligner based upon the real-time three-dimensional digital model.

In some embodiments, a method for automated dental aligner printing is disclosed. The method including the steps of: receiving a three-dimensional rendering of a user's teeth on a computer having a display screen, one or more processors, one or more transceivers for transmitting and receiving wireless data, one or more data storage devices and a means for scanning and uploading the three-dimensional rendering of a user's teeth, the computer executing a software program for automated dental aligner printing from a three-dimensional printer, the three-dimensional printer having a hot end, an extruder, a print bed and a cooling fan; generating a three-dimensional digital model of a dental aligner based upon the three-dimensional rendering of a user's teeth, on a computer having a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer; and printing a dental aligner based upon the real-time three-dimensional digital model from the three-dimensional printer, the three-dimensional printer having a hot end, an extruder, a print bed and a cooling fan, automatically and on the same day the three-dimensional rendering of a user's teeth is received by the computer.

The improved systems and methods described herein have many benefits and advantages over traditional systems and methods for producing dental aligners, including, but not limited to, increased convenience and efficiency for users, increased cost savings for producers of dental aligners, and increased access to quality dental services and products. Traditionally, clear aligners have been fabricated using a combination of milling and manual processes like trimming, resulting in a time-consuming and labor-intensive multi-day process involving multiple and different locations. The improved systems and methods described herein, including specifically the use of three-dimensional printing of the dental aligner accelerates the construction process by enabling a large batch of custom aligners to be produced directly from a patient's digital scans or physical mold at a self-serve kiosk, computer system and machine. Thus, providing direct patient access and real time production of custom fit dental aligners.

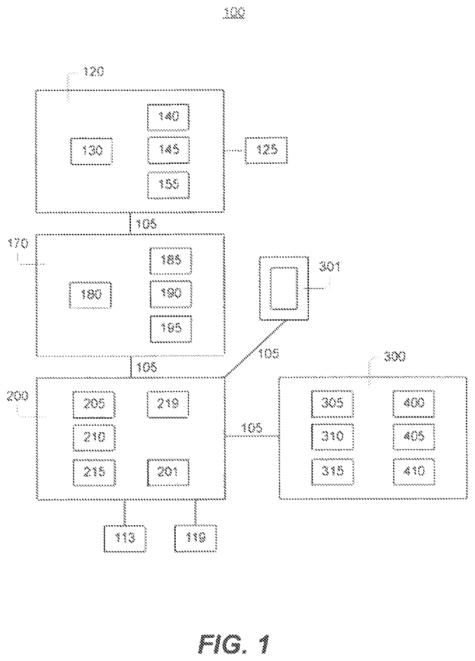

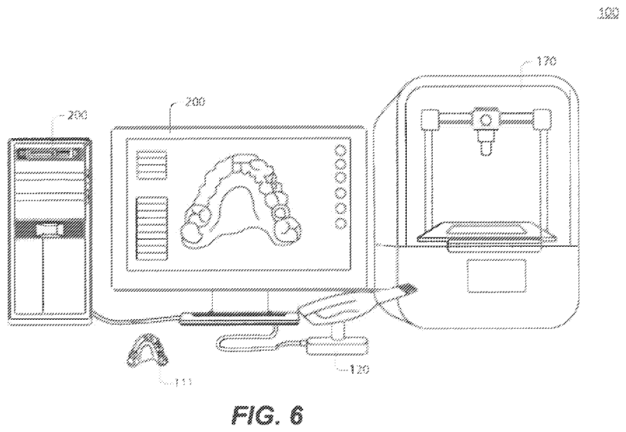

Referring now to FIGS. 1-3 and 6, shown therein are various embodiments of a system (100) for automated same-day dental scanning and dental aligner printing in accordance with the inventive concepts disclosed herein. In some embodiments the system (100) is configured to produce a dental aligner on the same day as the dental scan (i.e., production of the dental aligner is configured to be completed and delivered to a user in a single day from a single location); while in other embodiments the system (100) is configured to produce a dental aligner at a different day as the dental scan (i.e., production of the dental aligner may be completed or delivered to a user over the course of several days) for example. In an embodiment, the system (100) includes a three-dimensional printer (170) in communication with a computer (200) running one or more computer program (300). The three-dimensional printer (170) configured for automated same-day printing of a dental aligner (111) produced from a three-dimensional rendering (113) of a user's teeth. The three-dimensional printer (170) including a hot end (180), an extruder (185), a print bed (190) and a cooling fan (195). The computer (200) including a display screen (201), one or more processors (205), one or more transceivers (210) for transmitting and receiving wireless data (105), one or more data storage devices (215) and a means for scanning and uploading (219) the three-dimensional rendering (113) of a users teeth. In some embodiments the means for scanning and uploading (219) the three-dimensional rendering (113) of a user's teeth may also be located on the three-dimensional printer (170). The means for scanning and uploading (219) may be a combination of scanners, video cameras, computer or combinations thereof. The means for scanning and uploading (219) may be positioned within a shelf or opening of a housing of the system (100) such that a user can physically insert a physical mold or a pre-existing dental aligner directly into the shelf or opening of the housing and the physical mold or pre-existing dental aligner can be scanned by the means for scanning and uploading (219) to create a digital representation of the physical mold or pre-existing dental aligner (including any measurement data associated therewith) on one or more computer (200) for production of a new dental aligner (111). The computer (200) executing a software program (300) for automated same-day scanning of the three-dimensional rendering (113) of a users teeth and for automated same-day dental aligner printing from the three-dimensional printer (170). The software program (300) including automated instructions for: receiving (305) a three-dimensional rendering (113) of a user's teeth; generating (310) a three-dimensional digital model (119) of a dental aligner (111) based upon the three-dimensional rendering (113) of a user's teeth; and printing (315) from the three-dimensional printer (170), automatically and on the same day as the three-dimensional rendering (113) of a user's teeth is received, a dental aligner (111), based upon the real-time three-dimensional digital model (119).

In some embodiments, the system (100) also includes an intraoral scanner (120) in communication with the three-dimensional printer (170) and one or more computer (200) running one or more computer program (300). The intraoral scanner (120) for producing a real-time three-dimensional digital scan (125) of a user's teeth. The intraoral scanner (120) includes a scanner (130), one or more processors (140), one or more transceivers (145) for transmitting and receiving wireless data (105) and one or more data storage devices (155). The intraoral scanner (120) may include a variety of computers and software programs running in combination with the intraoral scanner (120) for example. The intraoral scanner (120) may be any type of intraoral scanner known in the art. For example, the intraoral scanner (120) may be physically connected or housed within the system (100) or may be a stand-alone piece or pieces of equipment in electronic communication with the system (100). For example, the intraoral scanner (120) may be a hand-held scanning wand, a fixed scanning booth, a wearable scanner (such as a headset or the like), or an intraoral scanner that is selectively attached and detached to a remote computer, mobile device, such as a mobile phone or other hand-held device and that is in electronic communication with the system (100).

Intraoral scanners are devices used for capturing direct optical impressions in dentistry. An intraoral scanner, such as intraoral scanner (120), projects a light source (such as a laser or structured light) onto the object to be scanned, typically the dental arches, including prepared teeth and implant scan bodies, such as cylinders screwed onto implants and used for transferring the three-dimensional position. The images produced by the intraoral scanner (120) may include images of dentogingival tissues as well as implant scan bodies captured by imaging sensors, which are then processed by the scanning software, including the software program (300) described herein, which generates point clouds. Point clouds are then triangulated by the same software, creating a three-dimensional surface model. The three-dimensional of the dentogingival tissues are the result of the optical impression and produces a digital scan, such as three-dimensional digital scan (125), which is used as a virtual alternative to traditional plaster molds.

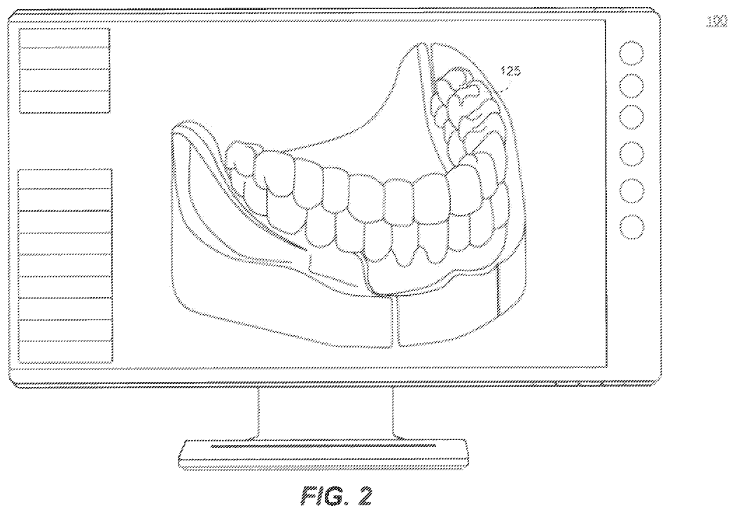

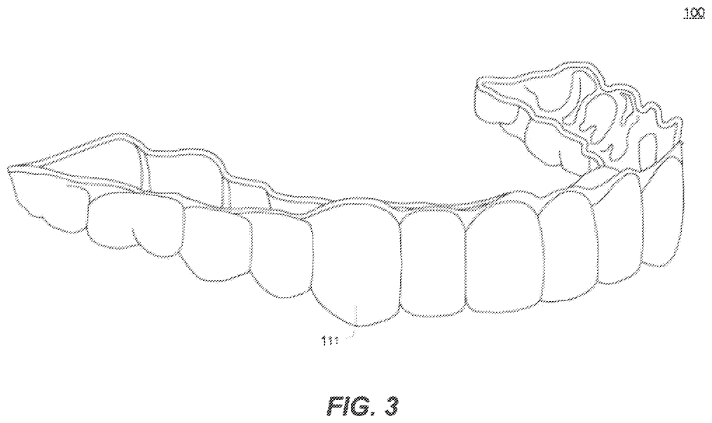

The three-dimensional printer (170) is in communication with the intraoral scanner (120). The three-dimensional printer (170) is for automated same-day printing of a dental aligner (111) (as shown in FIG. 3) produced from the real-time three-dimensional digital scan (125) of a users teeth (as shown in FIG. 2). The three-dimensional printer (170) includes a hot end (180), an extruder (185), a print bed (190) and a cooling fan (195), Three-dimensional printing, or additive manufacturing, is the construction of a three-dimensional object from a CAD model or a digital 3D model. Three-dimensional printing can refer to a variety of processes in which material is deposited, joined or solidified under computer control to create a three-dimensional object, with material being added together (such as plastics, liquids or powder grains being fused together), typically layer by layer. Three-dimensional printers, such as three-dimensional printer (170) use computer aided design software, such as software program (300) to create three-dimensional objects from a variety of materials, like plastics or powders. The three-dimensional printer works from the ground up, piling up layer upon layer of desired material until the printed object is produced, in this case the dental aligner (111). The three-dimensional printer (170) builds up a three-dimensional (“3D”) mod& one layer at a time, from the bottom upward, by repeatedly printing over the same area in a method known as fused depositional modeling (FDM). Working entirely autocratically, the three dimensional printer creates a model over a period of hours by turning a 3D computer scan of a custom fit dental aligner (111), based upon a user's three-dimensional digital scan or digital rendering of a physical mold, into lots of two-dimensional (“2D”), cross-sectional layers effectively separate 2D prints that sit one on top of another. Instead of using ink, which would never build up to much volume, the printer deposits layers of molten plastic or powder and fuses them together (and to the existing structure) with adhesive or ultraviolet light.

The system (100) further includes a computer (200) in communication with the intraoral scanner (120) and in communication the three-dimensional printer (170). The computer (200) having a display screen (201), one or more processors (205), one or more transceivers (210) for transmitting and receiving wireless data (105) and one or more data storage devices (215). The computer (200) executing a software program (300) for automated same-day, dental scanning from the intraoral scanner (120) and automated same-day dental aligner printing from the three-dimensional printer (170). The computer (200) is a computer or computers which may be housed or contained in any type of housing or device or as a stand-alone computer or computers or via multiple computers or servers interconnected through a wireless network. For example, a stand-alone desktop computer, mobile smart phone, tablet, laptop, or similar device for storing, sending, receiving and processing wireless data, according to instructions given to it by a computer program. Processors, transceivers, and data storage devices used in computers, such as desktop computers, laptops, mobile smart phones, tablets and the like are well known to persons of ordinary skill in the art and will not be described in further detail herein for purposes of brevity.

The software program (300) includes automated instructions for; receiving (305) a real-time three-dimensional digital scan (125) of a user's teeth from the intraoral scanner (120); generating (310) a three-dimensional digital model (119) of a dental aligner (111) based upon the real-time three-dimensional digital scan (125) of a user's teeth; and printing (315) from the three-dimensional printer (170), automatically and on the same day as the three-dimensional digital scan (125) of a user's teeth, a dental aligner (111) based upon the real-time three-dimensional digital model (119). In some embodiments, the software program (300) further includes automated instructions for: (d) determining (400) a treatment plan (405) for optimal positioning of the dental aligner (111) based upon artificial intelligence (410). Further, in some embodiments the treatment plan (405) for optimal positioning of the dental aligner (111) is determined by a license dentist or is approved by a licensed dentist (on the same day or at a later date) in combination with the software program (300) and one or more computer (200), In some embodiments, the licensed dentist may be available on-site, while in other embodiments the licensed dentist may be off-site but available via one or more computer (200) or video monitors, for example. In some embodiments, the computer (200) is in wireless communication with a mobile device (301) or other remote computer associated with a user. The computer (200) configured to send automatic notifications and alerts to the mobile device (301) or remote computer concerning the status, same day pick-up time, and cost associated with printing the dental aligner (111).

Artificial intelligence (“AI”), such as artificial intelligence (410) may be implemented with various embodiments of the inventive concepts disclosed herein. In some embodiments, for example, artificial intelligence may be used either in lieu of review and approval by a licensed dentist or in combination therewith, for example. Artificial intelligence is intelligence demonstrated by machines, unlike natural intelligence displayed by humans and animals, Artificial intelligence works by combining large amounts of data with fast, iterative processing and intelligent algorithms, allowing the software, such as software program (300) to learn automatically from patterns or features in the data. The process requires multiple passes at the data to find connections and derive meaning from undefined data. Artificial intelligence includes, a variety of methods and processes, including but not limited to machine learning, which automates analytical model building. It uses methods from neural networks, statistics, operations research and physics to find hidden insights in data without explicitly being programmed for where to look or what to conclude. Artificial intelligence also includes, a neural network, which is a type of machine learning that is made up of interconnected units (like neurons) that processes information by responding to external inputs, relaying information between each unit. The process requires multiple passes at the data to find connections and derive meaning from undefined data. Artificial intelligence also includes, deep learning, which uses huge neural networks with many layers of processing units, taking advantage of advances in computing power and improved training techniques to learn complex patterns in large amounts of data. Common applications include image and speech recognition. Artificial intelligence also includes, cognitive computing, which is a subfield that strives for a natural, human-like interaction with machines. Using artificial intelligence and cognitive computing, a machine can simulate human processes through the ability to interpret images and speech—and then speak coherently in response. Computer vision relies on pattern recognition and deep learning to recognize what's in a picture or video. When machines can process, analyze and understand images, they can capture images or videos in real time and interpret their surroundings. Natural language processing (NLP) is the ability of computers to analyze, understand and generate human language, including speech.

Computer software program code, such as computer program (300), for carrying out operations for the system (100) may be written in any combination of one or more programming languages such as Java, Smalltalk, C++ or the like and conventional procedural programming languages such as the “C” programming language or similar programming languages. The program code may execute entirely on the computer (200), or partly on the first computer (200) and partly on another remote computer (200), server, mobile smart phone application, or tablet for example.

In some embodiments, a variety of remote computers may be connected to the computer (200) or a second computer (200) through any type of network, public or private, cellular or the like and including a local area network (LAN) or a wide area network (WAN), or connection may be made to an external computer, such as through the internet on the world wide web using an internet service provider. The internet is a global computer network providing a variety of information and communications facilities, consisting of interconnected networks using standardized communications protocols, for example.

The computer program instructions may be provided to a processor of a computer, mobile smart phone, tablet, mobile computing device, general purpose computer, special purpose computer, or other programmable data processing apparatus to produce a machine and system, such that the instructions, which execute via the processor, the computer, mobile smart phone, tablet, or other mobile device create a means for implementing the functions and acts described herein. The computer program instructions may also be stored in a computer readable medium that can direct a computer, mobile computing device or other programmable data processing apparatus to function in a particular manner such that the instructions stored in the computer readable medium produce an article of manufacture including instructions which implement the functions and acts specified herein.

The computer program instructions may also be loaded directly onto a computer, mobile smart phone, mobile computing device, tablet or other programmable data processing apparatus to cause a series of operational steps to be performed on the computer, mobile smart phone, mobile computing device, tablet or other programmable data processing apparatus for implementing the functions or acts specified herein.

Data may be stored on the computer (200), a second computer (200), or on one or more remote computers (200), mobile smart phone devices, servers or other programmable data processing apparatuses. A computer readable storage medium may be, for example, an electronic, magnetic, optical, electromagnetic, infrared, or semiconductor system, apparatus or device or any suitable combination of the foregoing. An example of a computer readable storage medium includes an electrical connection having one or more wires, a portable computer diskette, hard disk, random access memory (RAM), erasable programmable read-only memory (EPROM), flash memory, CD-ROM, optical storage devices, magnetic storage devices, the like and any combination of the foregoing. By way of example, a computer readable storage medium is any medium or device that may contain or store a program for use by or in connection with an instruction execution system, apparatus or device. Likewise, data may be stored in the cloud or on one or more remote computers or servers or an interconnected network of computers or servers, for example.

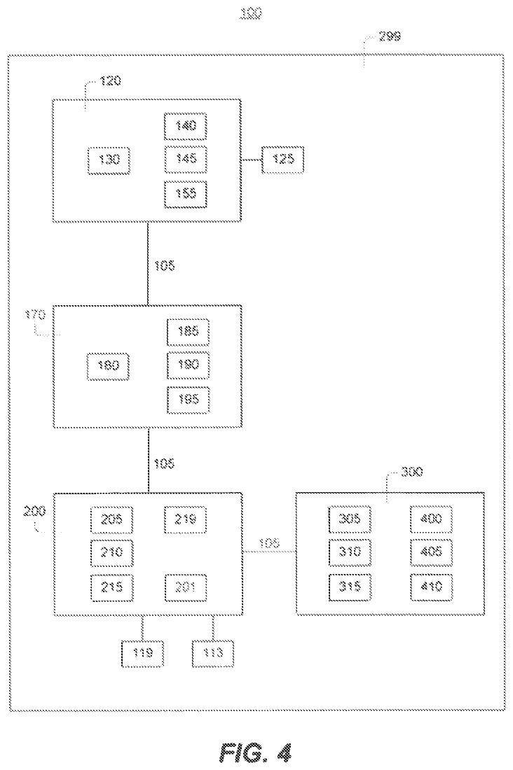

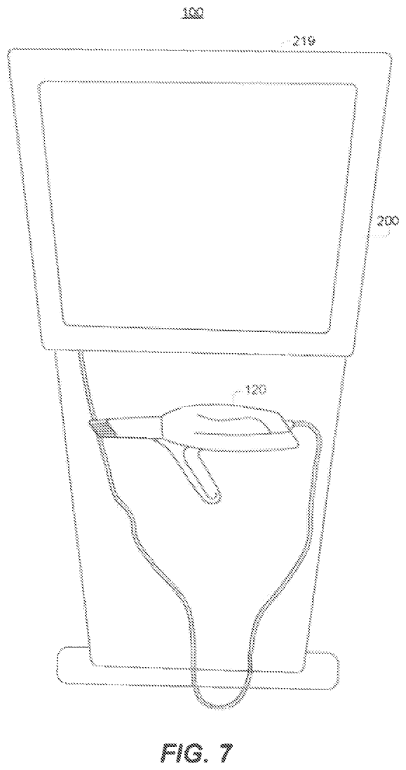

Referring now to FIGS. 4 and 7, shown therein are embodiments of a self-serve kiosk (299) in accordance with the inventive concepts disclosed herein. As described herein the self-serve kiosk (299) is a small structure or housing, containing a variety of computer and printing equipment, often free-standing, open on one or both sides and sometimes including a chair, bench or opening to facilitate self-serve use by a person. The self-serve kiosk (299) is configured to permit users to walk up to the kiosk, sit or stand in front of the self-serve kiosk (299) and have performed a three-dimensional digital scan of the person's teeth or scan and upload a pre-existing three dimensional rendering (113), such as a digital scan, x-ray, physical mold or impression and then to receive a custom fit dental aligner manufactured directly from the self-serve kiosk (299) in real time and on the same day. In some embodiments, the system (100), includes a computer (200) and a three-dimensional printer (170), while in other embodiments the system (100) also includes an intraoral scanner (120). In some embodiments, the system (100) includes a means for scanning and uploading (219). The means for scanning and uploading (219) may be scanners, video cameras, computers or combinations thereof. The means for scanning and uploading (219) may be positioned within a shelf or opening of a housing of the self-serve kiosk (299) such that a user can physically insert a traditional physical mold or a traditional pre-existing dental aligner directly into the shelf or opening of the housing and the physical mold or pre-existing dental aligner can be scanned by the means for scanning and uploading (219) to create a digital representation of the physical mold or pre-existing dental aligner (including any measurement data associated therewith) on one or more computer (200) for production of a new dental aligner (111). In some embodiments the computer (200) includes a means for scanning and uploading (219) a three-dimensional rendering (113). In some embodiments, each of the intraoral scanner (120), three-dimensional printer (170) and computer (200) are fully housed within the self-serve kiosk (299). While in other embodiments, the intraoral scanner (120) may be housed or located at a remote location or detachably connected to the system (100) and in wireless communication therewith. Further, in some embodiments of the system (100), the computer (200) is in wireless communication with one or more remote computer (200) associated with the self-serve kiosk (299).

As shown in FIGS. 4 and 7, the self-serve kiosk (299) is shown with an intraoral scanner (120) for producing a real-time three-dimensional digital scan (125) of a user's teeth. The intraoral scanner (120) including a scanner (130), one or more processors (140), one or more transceivers (145) for transmitting and receiving wireless data (105) and one or more data storage devices (155). As described above, intraoral scanners are devices used for capturing direct optical impressions in dentistry. An intraoral scanner, such as intraoral scanner (120), projects a light source (such as a laser or structured light) onto the object to be scanned, typically the dental arches, including prepared teeth and implant scan bodies, such as cylinders screwed onto implants and used for transferring the three-dimensional position. The images produced by the intraoral scanner (120) may include images of dentogingival tissues as well as implant scan bodies captured by imaging sensors, which are then processed by the scanning software, including the software program (300) described herein, which generates point clouds. Point clouds are then triangulated by the same software, creating a three-dimensional surface model. The three-dimensional of the dentogingival tissues are the result of the optical impression and produces a digital scan, such as three-dimensional digital scan (125), which is used as a virtual alternative to traditional plaster molds. The intraoral scanner (120) may be any type of intraoral scanner known in the art. Further, the intraoral scanner (120) may be physically connected or housed within the system (100) or may be a stand-alone piece or pieces of equipment in electronic communication with the system (100). For example, the intraoral scanner (120) may be a hand-held scanning wand, a fixed scanning booth, a wearable scanner (such as a headset and the like), or an intraoral scanner that is selectively attached and detached to a mobile phone or other hand-held device and that is in electronic communication with the system (100).

The self-serve kiosk (299) further including a three-dimensional printer (170) and computer (200) in communication with the intraoral scanner (120). In some embodiments, the three-dimensional printer (170) includes a means for scanning and uploading (219) a three-dimensional rendering (113) of a user's teeth. The means for scanning and uploading (219) may be scanners, video cameras, computers or combinations thereof for example. The means for scanning and uploading (219) may be positioned within a shelf or opening of a housing of the self-serve kiosk (299) such that a user can physically insert a traditional physical mold or a traditional pre-existing dental aligner directly into the shelf or opening of the housing and the physical mold or pre-existing dental aligner can be scanned by the means for scanning and uploading (219) to create a digital representation of the physical mold or pre-existing dental aligner (including any measurement data associated therewith) on one or more computer (200) for production of a new dental aligner (111). The computer (200) executing a software program (300) for automated same-day scanning of the three-dimensional rendering (113) of a user's teeth and for automated same-day dental aligner printing from the three-dimensional printer (170). The software program (300) including automated instructions for: receiving (305) a three-dimensional rendering (113) of a user's teeth; generating (310) a three-dimensional digital model (119) of a dental aligner (111) based upon the three-dimensional rendering (113) of a user's teeth; and (c) printing (315) from the three-dimensional printer (170), automatically and on the same day as the three-dimensional rendering (113) of a user's teeth is received, a dental aligner (111), based upon the real-time three-dimensional digital model (119).

In some embodiments, the three-dimensional printer (170) is configured for automated same-day printing of a dental aligner (111) produced from the real-time three-dimensional digital scan (125) of a user's teeth. The three-dimensional printer (170) including a hot end (180), an extruder (185), a print bed (190) and a cooling fan (195). The self-serve kiosk (299) further including a computer (200) in communication with the Intraoral scanner (120) and in communication with the three-dimensional printer (170). The computer (200) including a display screen (201), one or more processors (205), one or more transceivers (210) for transmitting and receiving wireless data (105) and one or more data storage devices (215). The computer (200) executing a software program (300) for automated same-day dental scanning from the intraoral scanner (120) and automated same-day dental angrier printing from the three-dimensional printer (170).

Three-dimensional printing, or additive manufacturing, is the construction of a three-dimensional object from a CAD model or a digital 3D model. Three-dimensional printing can refer to a variety of processes in which material is deposited, joined or solidified under computer control to create a three-dimensional object, with material being added together (such as plastics, liquids or powder grains being fused together), typically layer by layer. Three-dimensional printers, such as three-dimensional printer (170) use computer aided design software, such as software program (300) to create three-dimensional objects, such as a dental aligner (111) from a variety of materials, like plastics or powders. The three-dimensional printer works from the ground up, piling up layer upon layer of desired material until the printed object is produced, in this case the dental aligner (111). The three-dimensional printer (170) builds up a three-dimensional (“3D”) model one layer at a time, from the bottom upward, by repeatedly printing over the same area in a method known as fused depositional modeling (FDM). Working entirely automatically, the printer creates a model over a period of hours by turning a 30 image or model into lots of two-dimensional (“2D”), cross-sectional layers effectively separate 2D prints that sit one on top of another, but without the paper in between. Instead of using ink, which would never build up to much volume, the printer deposits layers of molten plastic or powder and fuses them together (and to the existing structure) with adhesive or ultraviolet light to form the dental aligner (111).

The software program (300) includes automated instructions for: receiving (305) a real-time three-dimensional digital scan (125) of a user's teeth from the intraoral scanner (120); generating (310) a three-dimensional digital model (119) of a dental aligner (111) based upon the real-time three-dimensional digital scan (125) of a user's teeth; and printing (315) from the three-dimensional printer (170), automatically and on the same day as the three-dimensional digital scan (125) of a user's teeth, a dental aligner (111) based upon the real-time three-dimensional digital model (119). In some embodiments, the software program (300) may include instructions for automated same-day scanning of the three-dimensional rendering (113) of a user's teeth and for automated same-day dental aligner printing from the three-dimensional printer (170). For example, the software program (300) may include automated instructions for: receiving (305) a three-dimensional rendering (113) of a user's teeth; generating (310) a three-dimensional digital model (119) of a dental aligner (111) based upon the three-dimensional rendering (113) of a user's teeth; and printing (315) from the three-dimensional printer (170), automatically and on the same day as the three-dimensional rendering (113) of a user's teeth is received, a dental aligner (111), based upon the real-time three-dimensional digital model (119).

Computer software program code, including software program (300), may include code for carrying out operations for the system (100) written in any combination of one or more programming languages such as Java, Smalltalk, C++ or the like and conventional procedural programming languages such as the “C” programming language or similar programming languages. The program code may execute entirely on the computer (200), or partly on the first computer (200) and partly on another remote computer (200), server, mobile smart phone application, or tablet for example.

In some embodiments, remote computers may be connected to the computer (200) or a second computer (200) through any type of network, public or private, cellular or the like and including a local area network (LAN) or a wide area network (WAN), or connection may be made to an external computer, such as through the internet on the world wide web using an internet service provider. The internet is a global computer network providing a variety of information and communications facilities, consisting of interconnected networks using standardized communications protocols, for example.

The computer program instructions may be provided to a processor of a computer, mobile smart phone, tablet, mobile computing device, general purpose computer, special purpose computer, or other programmable data processing apparatus to produce a machine and system, such that the instructions, which execute via the processor, the computer, mobile smart phone, tablet, or other mobile device create a means for implementing the functions and acts described herein. The computer program instructions may also be stored in a computer readable medium that can direct a computer, mobile computing device or other programmable data processing apparatus to function in a particular manner such that the instructions stored in the computer readable medium produce an article of manufacture including instructions which implement the functions and acts specified herein.

The computer program instructions may also be loaded directly onto a computer, mobile smart phone, mobile computing device, tablet or other programmable data processing apparatus to cause a series of operational steps to be performed on the computer, mobile smart phone, mobile computing device, tablet or other programmable data processing apparatus for implementing the functions or acts specified herein.

Data may be stored on the computer (200), a second computer (200), or on one or more remote computers (200), mobile smart phone devices, servers or other programmable data processing apparatuses. A computer readable storage medium may be, for example, an electronic, magnetic, optical, electromagnetic, infrared, or semiconductor system, apparatus or device or any suitable combination of the foregoing. An example of a computer readable storage medium includes an electrical connection having one or more wires, a portable computer diskette, hard disk, random access memory (RAM), erasable programmable read-only memory (EPROM), flash memory, CD-ROM, optical storage devices, magnetic storage devices, the like and any combination of the foregoing. By way of example, a computer readable storage medium is any medium or device that may contain or store a program for use by or in connection with an instruction execution system, apparatus or device. Likewise, data may be stored in the cloud or on one or more remote computers or servers or an interconnected network of computers or servers, for example.

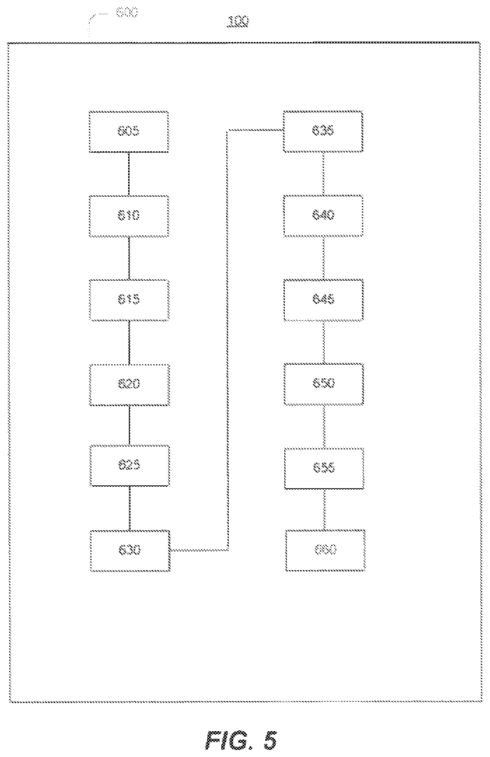

Referring now to FIG. 5, shown therein is an embodiment of a method (600) for automated same-day dental scanning and dental aligner printing. In some embodiments the method (600) is configured to produce a dental aligner on the same day as the dental scan (i.e., production of the dental aligner is configured to be completed and delivered to a user in a single day from a single location); while in other embodiments the method (600) is configured to produce a dental aligner at a different day as the dental scan (i.e., production of the dental aligner may be completed or delivered to a user over the course of several days) for example. In some embodiments, the method (600) includes the steps of: receiving (605) a three-dimensional rendering (113) of a user's teeth on a computer (200) having a display screen (201), one or more processors (205), one or more transceivers (210) for transmitting and receiving wireless data (105), one or more data storage devices (215) and a means for scanning and uploading (219) the three-dimensional rendering (113) of a user's teeth, the computer (200) executing a software program (300) for automated dental aligner (111) printing from a three-dimensional printer (170), the three-dimensional printer (170) having a hot end (180), an extruder (185), a print bed (190) and a cooling fan (195); generating (610) a three-dimensional digital model (119) of a dental aligner (111) based upon the three-dimensional rendering (113) of a user's teeth, on a computer (200) having a display screen (201), one or more processors (205), one or more transceivers (210) for transmitting and receiving wireless data (105) and one or more data storage devices (215), the computer (200) executing a software program (300) for automated same-day dental scanning from the intraoral scanner (120) and automated same-day dental aligner printing from the three-dimensional printer (170); and printing (615) a dental aligner (111) based upon the real-time three-dimensional digital model (119) from the three-dimensional printer (170), the three-dimensional printer (170) having a hot end (180), an extruder (185), a print bed (190) and a cooling fan (195), automatically and on the same day the three-dimensional rendering (113) of a user's teeth is received by the computer (200).

As described above, intraoral scanners are devices used for capturing direct optical impressions in dentistry. An intraoral scanner, such as intraoral scanner (120), projects a light source (such as a laser or structured light) onto the object to be scanned, typically the dental arches, including prepared teeth and implant scan bodies, such as cylinders screwed onto implants and used for transferring the three-dimensional position. The images produced by the intraoral scanner (120) may include images of dentogingival tissues as well as implant scan bodies captured by imaging sensors, which are then processed by the scanning software, including the software program (300) described herein, which generates point clouds. Point clouds are then triangulated by the same software, creating a three-dimensional surface model. The three-dimensional of the dentogingival tissues are the result of the optical impression and produces a digital scan, such as three-dimensional digital scan (125), which is used as a virtual alternative to traditional plaster molds. The means for scanning and uploading (219) may be scanners, video cameras, computers or combinations thereof for example. The means for scanning and uploading (219) may be positioned within a shelf or opening of a housing of the system (100) or self-serve kiosk (299) such that a user can physically insert a traditional physical mold or a traditional pre-existing dental aligner directly into the shelf or opening of the housing and the physical mold or pre-existing dental aligner can be scanned by the means for scanning and uploading (219) to create a digital representation of the physical mold or pre-existing dental aligner (including any measurement data associated therewith) on one or more computer (200) for production of a new dental aligner (111).

Three-dimensional printing, or additive manufacturing, is the construction of a three-dimensional object from a CAD model or a digital 3D model. Three-dimensional printing can refer to a variety of processes in which material is deposited, joined or solidified under computer control to create a three-dimensional object, with material being added together (such as plastics, liquids or powder grains being fused together), typically layer by layer. Three-dimensional printers, such as three-dimensional printer (170) use computer aided design software, such as software program (300) to create three-dimensional objects from a variety of materials, like plastics or powders. The three-dimensional printer (170) works from the ground up, piling up layer upon layer of desired material until the printed object is produced, in this case the dental aligner (111). The three-dimensional printer (170) builds up a three-dimensional (“3D”) model one layer at a time, from the bottom upward, by repeatedly printing over the same area in a method known as fused depositional modeling (FDM). Working entirely automatically, the printer creates a model over a period of hours by turning a 3D model into lots of two-dimensional (“2D”), cross-sectional layers effectively separate 2D prints that sit one on top of another. Instead of using ink, which would never build up to much volume, the printer deposits layers of molten plastic or powder and fuses them together (and to the existing structure) with adhesive or ultraviolet light to form the dental aligner (111).

Computer software program code for carrying out operations for the system (100) and software program (300) may be written in any combination of one or more programming languages such as Java, Smalltalk, C++ or the like and conventional procedural programming languages such as the “C” programming language or similar programming languages. The program code may execute entirely on the computer (200), or partly on the first computer (200) and partly on another remote computer (200), server, mobile smart phone application, or tablet for example.

In some embodiments, the method (600) includes the steps of: receiving (620) on a computer comprising a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a real-time three-dimensional digital scan of a user's teeth from an intraoral scanner, the intraoral scanner comprising, one or more processors, one or more transceivers for transmitting and receiving wireless data and one or more data storage devices; generating (625) on a computer comprising a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a three-dimensional digital model of a dental aligner based upon the real-time three-dimensional digital scan of a user's teeth; and printing (630) from a three-dimensional printer comprising a hot end, an extruder, a print bed and a cooling fan, automatically and on the same day as the three-dimensional digital scan of a user's teeth, a dental aligner based upon the real-time three-dimensional digital model.

Remote computers may be connected to the computer (200) or a second computer (200) through any type of network, public or private, cellular or the like and including a local area network (LAN) or a wide area network (WAN), or connection may be made to an external computer, such as through the internet on the world wide web using an internet service provider. The internet is a global computer network providing a variety of information and communications facilities, consisting of interconnected networks using standardized communications protocols, for example.

The computer program instructions may be provided to a processor of a computer, mobile smart phone, tablet, mobile computing device, general purpose computer, special purpose computer, or other programmable data processing apparatus to produce a machine and system, such that the instructions, which execute via the processor, the computer, mobile smart phone, tablet, or other mobile device create a means for implementing the functions and acts described herein. The computer program instructions may also be stored in a computer readable medium that can direct a computer, mobile computing device or other programmable data processing apparatus to function in a particular manner such that the instructions stored in the computer readable medium produce an article of manufacture including instructions which implement the functions and acts specified herein.

The computer program instructions may also be loaded directly onto a computer, mobile smart phone, mobile computing device, tablet or other programmable data processing apparatus to cause a series of operational steps to be performed on the computer, mobile smart phone, mobile computing device, tablet or other programmable data processing apparatus for implementing the functions or acts specified herein.

Data may be stored on the computer (200), a second computer (200), or on one or more remote computers (200), mobile smart phone devices, servers or other programmable data processing apparatuses. A computer readable storage medium may be, for example, an electronic, magnetic, optical, electromagnetic, infrared, or semiconductor system, apparatus or device or any suitable combination of the foregoing. An example of a computer readable storage medium includes an electrical connection having one or more wires, a portable computer diskette, hard disk, random access memory (RAM), erasable programmable read-only memory (EPROM), flash memory, OD-ROM, optical storage devices, magnetic storage devices, the like and any combination of the foregoing. By way of example, a computer readable storage medium is any medium or device that may contain or store a program for use by or in connection with an instruction execution system, apparatus or device. Likewise, data may be stored in the cloud or on one or more remote computers or servers or an interconnected network of computers or servers, for example.

In some embodiments, the method (600) includes the step of: generating (635) on a computer (200) including a display screen (201), one or more processors (205), one or more transceivers (210) for transmitting and receiving wireless data (105) and one or more data storage devices (155), the computer (200) executing a software program (300) for automated same-day dental scanning from the intraoral scanner (120) and automated same-day dental aligner printing from the three-dimensional printer (170), a three-dimensional digital model (119) of a dental aligner (111) based upon the real-time three-dimensional digital scan (125) of a user's teeth. The method (600) further including the step of: printing (640) from a three-dimensional printer (170) including a hot end (180), an extruder (185), a print bed (190) and a cooling fan (195), automatically and on the same day as the three-dimensional digital scan (125) of a user's teeth, a dental aligner (111) based upon the real-time three-dimensional digital model (119).

In some embodiments, the method (600) includes the step of: generating (645) on a computer (200) including a display screen (201), one or more processors (205), one or more transceivers (210) for transmitting and receiving wireless data (105) and one or more data storage devices (155), the computer (200) executing a software program (300) for automated same-day dental scanning from the intraoral scanner (120) and automated same-day dental aligner printing from the three-dimensional printer (170), a treatment plan (405) for optimal positioning of the dental aligner (111) based upon artificial intelligence (410). Further, in some embodiments, the method (600) includes the step of: sending (650) from a computer (200) including a display screen (201), one or more processors (205), one or more transceivers (210) for transmitting and receiving wireless data (105) and one or more data storage devices (155), the computer (200) executing a software program (300) for automated same-day dental scanning from the intraoral scanner (120) and automated same-day dental aligner printing from the three-dimensional printer (170), a real-time notification that the dental aligner (111) has been printed from the three-dimensional printer (170) and is ready for same-day pick-up, to a mobile device (301) associated with a user.

In some embodiments, the method (600) includes the step of: displaying (655) on a display screen (201) of a computer (200) including one or more processors (205), one or more transceivers (210) for transmitting and receiving wireless data (105) and one or more data storage devices (155), the computer (200) executing a software program (300) for automated same-day dental scanning from the intraoral scanner (120) and automated same-day dental aligner printing from the three-dimensional printer (170), a three-dimensional digital model (119) of the dental aligner (111) based upon the real-time three-dimensional digital scan (125) of a user's teeth. Further, in some embodiments, the method (600) includes the step of: calculating (660) on a computer (200) including a display screen (201), one or more processors (205), one or more transceivers (210) for transmitting and receiving wireless data (105) and one or more data storage devices (155), the computer (200) executing a software program (300) for automated same-day dental scanning from the intraoral scanner (120) and automated same-day dental aligner printing from the three-dimensional printer (170), based upon artificial intelligence (410), optimal positioning of the dental aligner (111) in a mouth of a user.

Artificial intelligence (“AI”), including artificial intelligence (410) is intelligence demonstrated by machines, unlike natural intelligence displayed by humans and animals. Artificial intelligence may be implemented with various embodiments of the inventive concepts disclosed herein. In some embodiments, for example, artificial intelligence may be used either in lieu of review and approval by a licensed dentist or in combination therewith, for example. Artificial intelligence works by combining large amounts of data with fast, iterative processing and intelligent algorithms, allowing the software, such as software program (300) to learn automatically from patterns or features in the data. The process requires multiple passes at the data to find connections and derive meaning from undefined data, Artificial intelligence includes, a variety of methods and processes, including but not limited to machine learning, which automates analytical model building. It uses methods from neural networks, statistics, operations research and physics to find hidden insights in data without explicitly being programmed for where to look or what to conclude. Artificial intelligence also includes, a neural network, which is a type of machine learning that is made up of interconnected units (like neurons) that processes information by responding to external inputs, relaying information between each unit. The process requires multiple passes at the data to find connections and derive meaning from undefined data. Artificial intelligence also includes, deep learning uses huge neural networks with many layers of processing units, taking advantage of advances in computing power and improved training techniques to learn complex patterns in large amounts of data. Common applications include image and speech recognition. Artificial intelligence also includes, cognitive computing, which is a subfield that strives for a natural, human-like interaction with machines. Using artificial intelligence and cognitive computing, a machine can simulate human processes through the ability to interpret images and speech—and then speak coherently in response, for example. Computer vision relies on pattern recognition and deep learning to recognize what's in a picture or video. When machines can process, analyze and understand images, they can capture images or videos in real time and interpret their surroundings. Natural language processing (NLP) is the ability of computers to analyze, understand and generate human language, including speech.

In some embodiments, a method of automated dental scanning and dental aligner printing is also disclosed. The method includes the steps of: providing an intraoral scanner to a user, the intraoral scanner comprising one or more processors, one or more transceivers for transmitting and receiving wireless data and one or more data storage devices; receiving on the intraoral scanner, a real-time three-dimensional digital scan of the user's teeth; receiving the real-time three-dimensional digital scan of the user's teeth; printing a dental aligner from a three-dimensional printer, the dental aligner based upon the three-dimensional digital scan of the user's teeth; and providing the printed dental aligner to the user. In some embodiments of the method, the intraoral scanner is mailed to the user with instructions for use, such that the user can perform the intraoral scan at home; while in other embodiments, the user may schedule a time to have a professional, such as a licensed dentist or other dental professional visit the users home to perform the intraoral scan. Once the digital scan is complete, the intraoral scanner may be mailed to a designated location for printing of the dental aligner and then the printed dental aligner may be mailed to the user's home within twenty-four to forty-eight hours, for example. In some embodiments, after a digital scan is made of the user's teeth using the intraoral scanner, the user may elect to travel to a self-serve kiosk (as described herein) where the intraoral scanner and or the three-dimensional digital scan of the user's teeth may be returned and uploaded to the kiosk for direct printing of the dental aligner from the three-dimensional printer associated with the kiosk. The dental aligner may be printed at the kiosk and picked up by the user within twenty-four to forty-eight hours for example, or the user may elect to have the dental aligner mailed directly to the user's home, or the user may schedule printing of the dental aligner for a certain date/time such that the dental aligner may be picked up or delivered to the user at a date and time selected by the user.

It should be understood that embodiments of the inventive concepts disclosed herein may be offered, shipped or sold separately or together via one or more websites, as a system, a software program, or programs, or pre-loaded on various systems, hardware, kiosks, computers or mobile devices, including external hard drives, such as CD-ROM's, thumb-drives and the like for example. Further, it should be understood that the inventive concepts disclosed herein, including, for example, the systems, methods, kiosks, computers, printers, scanners and other equipment described herein may be fully or partially contained or housed within one or more vehicle or other mobile carrier, such as a van, backpack, aircraft or the like so that the systems and methods described herein may be brought directly to persons (who may be unable or unwilling to travel) or made available to persons at various remote locations. In this way, the efficiency and convenience of the inventive concepts disclosed herein is enhanced and access to dental treatment and custom dental aligners is increased, improving the health and well-being of those that receive the treatment and care.

From the above description, it is clear that the inventive concepts disclosed herein are adapted to carry out the objects and to attain the advantages mentioned herein as well as those inherent in the inventive concepts disclosed herein. While exemplary embodiments of the inventive concepts disclosed herein have been described for purposes of this disclosure, it will be understood that numerous changes may be made which will readily suggest themselves to those skilled in the art and which are accomplished within the broad scope of the inventive concepts disclosed herein and defined by the appended claims.

What is claimed is:

1. A system for automated dental aligner printing, comprising:
a three-dimensional printer, the three-dimensional printer for automated same-day printing of a dental aligner produced from a three-dimensional rendering of a user's teeth, the three-dimensional printer comprising a hot end, an extruder, a print bed and a cooling fan; and
a computer in communication with the three-dimensional printer, the computer comprising a display screen, one or more processors, one or more transceivers for transmitting and receiving wireless data, one or more data storage devices and a means for scanning and uploading the three-dimensional rendering of a user's teeth, the computer executing a software program for automated same-day scanning of the three-dimensional rendering of a user's teeth and for automated same-day dental aligner printing from the three-dimensional printer, the software program comprising automated instructions for:
receiving a three-dimensional rendering of a user's teeth;
generating a three-dimensional digital model of a dental aligner based upon the three-dimensional rendering of a user's teeth; and
printing from the three-dimensional printer, automatically and on the same day as the three-dimensional rendering of a user's teeth is received, a dental aligner, based upon the real-time three-dimensional digital model.

2. A method for automated dental aligner printing, the method comprising the steps of:
receiving a three-dimensional rendering of a user's teeth on a computer comprising a display screen, one or more processors, one or more transceivers for transmitting and receiving wireless data, one or more data storage devices and a means for scanning and uploading the three-dimensional rendering of a user's teeth, the computer executing a software program for automated dental aligner printing from a three-dimensional printer, the three-dimensional printer comprising a hot end, an extruder, a print bed and a cooling fan;
generating a three-dimensional digital model of a dental aligner based upon the three-dimensional rendering of a user's teeth, on a computer comprising a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer; and
printing a dental aligner based upon the real-time three-dimensional digital model from the three-dimensional printer, the three-dimensional printer comprising a hot end, an extruder, a print bed and a cooling fan, automatically and on the same day the three-dimensional rendering of a user's teeth is received by the computer.

3. A system for automated same-day dental scanning and dental aligner printing, comprising:
an intraoral scanner for producing a real-time three-dimensional digital scan of a user's teeth, the intraoral scanner comprising, a scanner, one or more processors, one or more transceivers for transmitting and receiving wireless data and one or more data storage devices;
a three-dimensional printer in communication with the intraoral scanner, the three-dimensional printer for automated same-day printing of a dental aligner produced from the real-time three-dimensional digital scan of a user's teeth, the three-dimensional printer comprising a hot end, an extruder, a print bed and a cooling fan;
a computer in communication with the intraoral scanner and in communication the three-dimensional printer, the computer comprising a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer; the software program comprising automated instructions for:
receiving a real-time three-dimensional digital scan of a user's teeth from the intraoral scanner;
generating a three-dimensional digital model of a dental aligner based upon the real-time three-dimensional digital scan of a user's teeth; and
printing from the three-dimensional printer, automatically and on the same day as the three-dimensional digital scan of a user's teeth, a dental aligner based upon the real-time three-dimensional digital model.

4. The system of claim 3, wherein the software program for automated same-day dental scanning from the intraoral scanner and automated same-day dental aligner printing from the three-dimensional printer, further comprises automated instructions for:
determining a treatment plan for optimal positioning of the dental aligner based upon artificial intelligence.

5. The system of claim 3, wherein the intraoral scanner, three-dimensional printer and computer are each physically housed within a self-serve kiosk.

6. The system of claim 3, wherein the computer is in wireless communication with a mobile device associated with a user, the computer configured to send automatic notifications and alerts to the mobile device concerning the status, same day pickup time, and cost associated with printing the dental aligner.

7. The system of claim 3, wherein the computer is in wireless communication with a remote computer associated with a self-serve kiosk, the self-serve kiosk comprising:
an intraoral scanner for producing a real-time three-dimensional digital scan of a user's teeth, the intraoral scanner comprising, one or more processors, one or more transceivers for transmitting and receiving wireless data and one or more data storage devices;
a three-dimensional printer in communication with the intraoral scanner, the three-dimensional printer for automated same-day printing of a dental aligner produced from the real-time three-dimensional digital scan of a user's teeth, the three-dimensional printer comprising a hot end, an extruder, a print bed and a cooling fan;
a computer in communication with the intraoral scanner and in communication the three-dimensional printer, the computer comprising a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, the software program comprising automated instructions for:
receiving a real-time three-dimensional digital scan of a user's teeth from the intraoral scanner;
generating a three-dimensional digital model of a dental aligner based upon the real-time three-dimensional digital scan of a user's teeth; and
printing from the three-dimensional printer, automatically and on the same day as the three-dimensional digital scan of a user's teeth, a dental aligner based upon the real-time three-dimensional digital model.

8. A method for automated same-day dental scanning and dental aligner printing, the method comprising the steps of:
receiving on a computer comprising a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a real-time three-dimensional digital scan of a user's teeth from an intraoral scanner, the intraoral scanner comprising, one or more processors, one or more transceivers for transmitting and receiving wireless data and one or more data storage devices;
generating on a computer comprising a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a three-dimensional digital model of a dental aligner based upon the real-time three-dimensional digital scan of a user's teeth; and
printing from a three-dimensional printer comprising a hot end, an extruder, a print bed and a cooling fan, automatically and on the same day as the three-dimensional digital scan of a user's teeth, a dental aligner based upon the real-time three-dimensional digital model.

9. The method of claim 8 further comprising the step of:
generating on a computer comprising a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a treatment plan for optimal positioning of the dental aligner based upon artificial intelligence.

10. The method of claim 8 further comprising the step of:
sending from a computer comprising a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a real-time notification that the dental aligner has been printed from the three-dimensional printer and is ready for same-day pick-up, to a mobile device associated with a user.

11. The method of claim 8 further comprising the step of:
displaying on a display screen of a computer comprising a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, a three-dimensional digital model of the dental aligner based upon the real-time three-dimensional digital scan of a user's teeth.

12. The method of claim 8 further comprising the step of:
calculating on a computer comprising a display screen, one or more processors, one or more transceivers for transmitting and receiving wireless data and one or more data storage devices, the computer executing a software program for automated same-day dental scanning from the intraoral scanner and automated same-day dental aligner printing from the three-dimensional printer, based upon artificial intelligence, optimal positioning of the dental aligner in a mouth of a user.

13. A method of automated dental scanning and dental aligner printing comprising the steps of:
providing an intraoral scanner to a user, the intraoral scanner comprising one or more processors, one or more transceivers for transmitting and receiving wireless data and one or more data storage devices;
receiving on the intraoral scanner, a real-time three-dimensional digital scan of the user's teeth;
receiving the real-time three-dimensional digital scan of the user's teeth;
printing a dental aligner from a three-dimensional printer, the dental aligner based upon the three-dimensional digital scan of the user's teeth; and
providing the printed dental aligner to the user.
